(19) United States
(12) Patent Application Publication
(10) Pub. No.: US 2014/0228950 A1
(43) Pub. Date: Aug. 14, 2014

(54) METHODS FOR AUGMENTING OR RECONSTRUCTING A HUMAN BREAST

(71) Applicant: ALLERGAN, INC., Irvine, CA (US)

(72) Inventors: Scott Whitcup, Laguna Hills, CA (US); David Schuessler, Ventura, CA (US)

(73) Assignee: ALLERGAN, INC., Irvine, CA (US)

(21) Appl. No.: 13/767,693

(22) Filed: Feb. 14, 2013

(51) Int. Cl. A61F 2/12

(52) U.S. Cl. CPC A61F 2/12
USPC 623/8

(57) ABSTRACT

Breast reconstruction or augmentation methods are provided which generally include implanting a mammary prosthesis comprising a shell having a self sealing wall and defining an inflatable cavity, inflating the prosthesis by introducing a fluid into the cavity using a cannula inserted percutaneously into the self sealing wall, imaging the procedure to assist with guiding the needle, and repeating the steps of inflating, imaging and allowing tissue and skin to expand, until a desired aesthetic outcome is achieved.

The present invention generally relates to medical implants and more specifically relates to inflatable breast prostheses and methods of reconstructing or augmenting a human breast.

Prostheses or implants for reconstruction and/or augmentation of the human body are well known.

Fluid filled prostheses, for example, mammary prostheses or breast implants, are widely used to replace excised tissue, for example after a radical mastectomy, or to augment the body to change the size or shape of the female breast.

During a mastectomy, a surgeon often removes skin as well as breast tissue, leaving the remaining chest tissues flat and tight. Woman often choose to undergo breast reconstruction to restore a more normal appearance to the breast, by having a breast prosthesis implanted. Often, the reconstruction surgery is not performed immediately after mastectomy, for various medical reasons. More typically, the reconstruction is done some time after the patient has healed from the mastectomy. This type of breast reconstruction is commonly referred to as a two-stage reconstruction or two-stage delayed reconstruction.

To create a breast-shaped space for a reconstructive implant, a tissue expander is commonly used to gradually expand the skin and tissue to make room for a permanent implant. An implanted tissue expander, which is like an inflatable balloon, is placed under the skin, remaining breast tissue, and possibly the chest muscle. Through a valve connected by a conduit to the tissue expander, the valve being, for example, implanted under the skin near the arm pit, the surgeon injects a saline solution at regular intervals to fill the expander over time, typically, for about 4 to 6 months. After the skin over the breast area has been sufficiently expanded, a second surgery is done to remove the expander and the permanent breast prosthesis is then implanted into the cavity left behind by the tissue expander.

Breast augmentation surgery (augmentation mammoplasty) is one of the most common cosmetic surgical procedures in the world, and is typically done by placing silicone or saline filled breast implants into normal healthy breast tissue. Augmentation is done primarily to correct cosmetic defects, asymmetry of the breasts, or enhance breast size, form, and/or tone. Conventional breast implants are designed to remain in the body for a lifetime, or at least up to ten years, or more.

When a woman is unsatisfied with the outcome of her augmentation surgery, for example, her breasts are not as large as she would like, or conversely, are much larger than what she had originally desired, she typically has no other option than to have the implants removed and replaced. In other instances, asymmetry of the breasts after augmentation is discovered, which is not obvious at the time of implant due to swelling of the tissue immediately post surgery. For these and other reasons, it is not uncommon for some women to undergo one or more breast augmentation revision surgeries months or years after the original surgery, in order to reach a perfect cosmetic outcome. However, breast augmentation is still considered a major surgery, and thus the additional cost, discomfort, pain, as well as the potential for complications that are part of any major surgical decision, must be carefully weighed and considered. Thus, naturally, it would be highly advantageous to provide a breast implant that could be size-adjusted in situ, with little or no surgical intervention, as is provided by the present invention.

Bark et al., U.S. Pat. No. 5,066,303 discloses a self-sealing tissue expander with a shell having a flowable sealing material.

Becker, U.S. Pat. No. 7,081,136, incorporated in its entirety herein by this specific reference, discloses an adjustable gel-filled mammary implant including inner and outer envelopes with the outer envelope containing a silicone gel and an inner envelope attached to the outer envelope filled with saline. The implant includes a self-sealing valve, filling tube and mini-reservoir for adjusting the size of the implant. A method for implanting, adjusting the size and removal of the filling tube and mini-reservoir are also disclosed.

Schuessler, U.S. patent application Ser. No. 12/543,795, filed on Aug. 19, 2009, and commonly owned with the present application, the entire disclosure of which is incorporated herein by this specific reference, discloses a fluid filled implant including a self-sealing shell.

There is a still a need for improved breast prostheses and methods for augmenting or reconstructing a female breast.

### SUMMARY OF THE INVENTION

The invention relates to expandable, adjustable prostheses, for example, breast implants and tissue expanders, and in particular to implantable devices that function as both temporary adjustable implants as well as adjustable permanent mammary prostheses. Methods for augmenting or reconstructing a breast using adjustable breast implants are also provided.

It is to be appreciated that the terms “implant” “prosthesis” and “tissue expander” as used herein are intended to encompass permanent implants, including adjustable implants, as well as relatively temporary tissue expanders, and components, for example, shells, of such implantable devices.

In one aspect of the invention, inflatable prosthetic implants are provided which include, as a component of such implants, self sealing shells, or shell walls that self seal around a needle puncture. For example, the implants comprise an elastomeric envelope or shell, defining a cavity for containing a fluid such as a saline solution. The shell may be partially or entirely self-sealing, in that once punctured by a needle, the puncture self seals such that leaking of fluid from the cavity, or tearing of the shell wall is prevented. In one aspect of the invention, the implants do not include a fill valve, and can be filled with fluid such as saline, using a needle or cannula inserted at any location of the self sealing wall, which may make up nearly the entirety of the shell.

In one aspect of the invention, a method of using an inflatable device or prosthesis for augmenting or reconstructing a breast is provided. The method generally comprises the steps of providing a prosthesis implanted in a breast, and imaging the breast while the prosthesis is being inflated with fluid. In some embodiments, computational image guidance techniques are employed to facilitate guiding of a distal end of a needle into the cavity of the implant.

Each and every feature described herein, and each and every combination of two or more of such features, is included within the scope of the present invention provided that the features included in such a combination are not mutually inconsistent.

### BRIEF DESCRIPTION OF THE DRAWING

The present invention may be more clearly understood and certain aspects and advantages thereof better appreciated with reference to the following Detailed Description when considered with the accompanying Drawing of which:

FIG. 1 is cross-sectional view of a adjustable breast prosthesis in accordance with an embodiment of the invention, the prosthesis shown as implanted in a breast of a human being and being inflated using methods of the invention.

### DETAILED DESCRIPTION

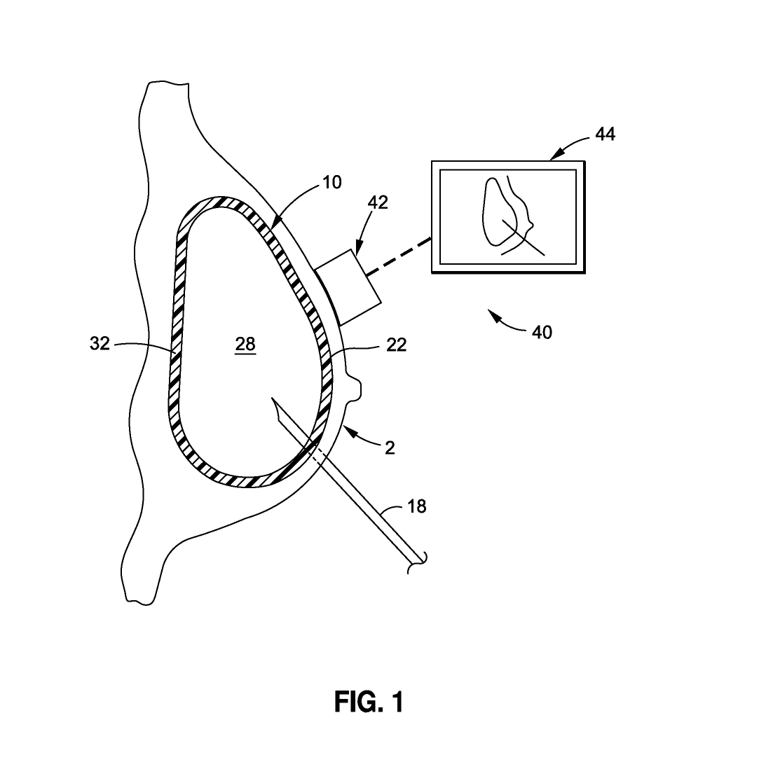

Turning now to FIG. 1, an inflatable breast prosthesis, in accordance with one embodiment of the invention, is shown generally at 10, as implanted in a human breast 2. The device 10 is being inflated with a suitable fluid, such as a saline solution, by means of a cannula coupled to a fluid source, for example, a needle 18 coupled to a syringe (not shown).

The prosthesis 10 generally comprises an flexible envelope or shell 22 defining a cavity 28 for containing a fluid.

The shell 22 may be made of any suitable material, preferably a self sealing material. The self sealing material is a material which will not maintain a puncture from a needle, and will, for example, create a fluid tight seal at a puncture location when a needle is withdrawn. This eliminates the need for a fluid fill port. Suitable materials are described, for example, in U.S. patent application Ser. No. 13/021,523, filed on Feb. 4, 2011, U.S. patent application Ser. No. 13/291,695, filed on Nov. 8, 2011, U.S. patent application Ser. No. 12/543,795, filed on Aug. 19, 2009, U.S. patent application Ser. No. 12/543,805 filed on Aug. 19, 2009, and U.S. patent application Ser. No. 13/105,715, filed on May 11, 2011, the entire disclosure of each of these patent applications being incorporated herein by this reference.

Inflation of the cavity 28 causes expansion of the prosthesis 10. The total volume of the device 10 is size adjustable by the introduction and removal of fluid into and from the fillable cavity 28.

In some embodiments, the prosthesis may include a portion, for example, a posterior portion 32, that is somewhat resistant to expansion, bending or buckling upon inflation of cavity 28. Thus, when inflated, the implant will properly expand anteriorly rather than in a spherical manner. In some embodiments, the posterior portion 32 is at least somewhat resistant to needle puncture, for example, may be puncture resistant, or even puncture proof.

In some embodiments, the entire implant shell 22 is penetratable by a sharp needle, and is self sealing to needle puncture, while in other embodiments, only an anterior portion of the implant shell is made of such a material. It is preferable, in some embodiments, that the entire shell 22, when substantially empty or not containing a fluid in the cavity, is foldable, rollable or otherwise quite pliable such that the implant can be introduced into a breast through a minimally sized incision.

The shell 22 may comprise an elastomeric material, for example, a silicone elastomer conventionally used for breast implants. In one embodiment, the shell comprises a dimethyl silicone elastomer, for example, a substantially homogeneous dimethyl-diphenyl silicone elastomer. One composition useful in the present invention is described in Schuessler, et al., U.S. application Ser. No. 12/179,340, filed Jul. 24, 2008, the disclosure of which is incorporated herein in its entirety by this specific reference. The elastomeric material may comprise a room temperature vulcanizing (RTV) or a high temperature vulcanizing (HTV) silicone from about 0.1-95 wt %, for example, about 1-40 wt %, for example, about 30 wt %. In an exemplary embodiment, the silicone-based fluid material is a high temperature vulcanizing (HTV) platinum-cured silicone dispersion in xylene.

The volume of the implant can be adjusted in situ by accessing the cavity 28 with a needle 18 through the self-sealing anterior portion of the device 10. In some embodiments, the shell 22 comprises layers of a silicone gel having a more flowable silicone gel disposed therebetween. Such a dual lumen shell is described for example, in U.S. patent application Ser. No. 13/105,715, filed on May 11, 2011. This embodiment provides a comfortable implant having the desirable qualities of a gel-filled implant with the advantages of being size-adjustable with saline.

For filling an implant of the present invention, syringe coupled to a cannula or needle 18, for example, a 21g or smaller needle, may be used. The needle may be introduced anywhere through the self sealing wall of the implant.

In some embodiments, the implant is filled with an aqueous solution, for example, a saline solution, a gel, for example a biocompatible hydrogel, such as a hyaluronic acid-based hydrogel, or a combination thereof. A suitable injection device including an appropriately sized needle/cannula, based on the type of fluid being injected, is used for inflating the implant in situ. For example, the injection device may comprise a manual syringe, or may include a motorized handpiece capable of controlling injection of hydrogel.

In one aspect, positioning of a distal end of the needle is facilitated using real-time imaging of the breast. Any suitable conventional imaging equipment 40 and techniques may be used, for example, computer guided imaging equipment and techniques.

Ultrasound, also known as sonography, uses high-frequency sound waves to outline a part of the body and to visualize internal organs and tissue. High-frequency sound waves are transmitted into the area of the body being studied and echoed back. A computer or dedicated electronic circuitry picks up the sound wave echoes and changes them into an image that is displayed on a computer screen. Breast ultrasound is sometimes used to evaluate breast abnormalities that are found during mammography or a physical exam. Ultrasound can be used as an imaging technique within the scope of the present invention. In general, non-intrusive/non-invasive acoustics inspection techniques utilizing ultrasound technology can be used to produce 3-D and 2-D images, using technologies known to those of skill in the art. It should be appreciated that acoustic inspection techniques utilizing other sound based pressure waves, such as infrasound waves or audible sound waves, can also be used to accomplish the imaging step.

FIG. 1 shows an ultrasound probe 42 and monitor 44 for viewing the breast and implant during the introduction of fluid into cavity 28. Advantageously, using imaging techniques, such as, but not limited to ultrasonic imaging, a sharp distal end of the needle 18 may be visually guided into the cavity of the prosthesis without contacting or penetrating the far wall of the prosthesis. Using the needle 18, the implant is then filled with saline or other biocompatible fluid, until the desired volume is reached. After removal of the needle, the prosthesis shell self-seals and prevents the implant from leaking.

In one embodiment of the invention, a method of augmenting or reconstructing a breast is provided, the method comprising the steps of providing a fillable breast prosthesis in a breast, the prosthesis comprising a flexible shell defining an internal cavity; imaging the breast having the prosthesis therein; introducing a cannula or needle through breast tissue and into the cavity of the prosthesis during the step of imaging; guiding the needle or cannula such that a distal tip of the needle or cannula is positioned in the cavity; and introducing into, or withdrawing a fluid from, the cavity through the distal tip of the needle or cannula during the step of imaging in order to adjust volume of the implant in the breast.

In one embodiment the imaging comprises fluoroscopy imaging. In another embodiment, the imaging comprises magnetic resonance imaging. In another embodiment, the imaging comprises ultrasonic imaging. In yet other embodiments, the imaging comprises mammography.

In yet another embodiment, a method for implanting an adjustable gel filled mammary prosthesis and for adjusting the size of the prosthesis after implantation is provided. The method may comprise the steps of providing a mammary prosthesis comprising a shell having a self sealing wall, the shell defining a cavity for containing a fluid; implanting the prosthesis into a breast of a patient; percutaneously introducing a distal end of a cannula through the self sealing wall; imaging the breast, prosthesis and cannula during the step of percutaneously introducing, to assist in guiding a position of the distal end of the cannula or needle in the cavity; introducing a fluid into the cavity through the distal end of the cannula during the step of imaging; and withdrawing the needle or cannula from the prosthesis and breast. The steps of introducing fluid may be repeated until the desired outcome is achieved.

In yet another embodiment, a breast reconstruction method for a patient who has undergone a mastectomy is provided, the method comprising the steps of implanting a mammary prosthesis comprising a shell having a self sealing wall, the shell defining an inflatable cavity; inflating the prosthesis by introducing a fluid into the cavity using a cannula inserted percutaneously into the self sealing wall of the implanted prosthesis; imaging the prosthesis during the step of inflating; allowing tissue/skin to expand in response to the inflated prosthesis; and repeating the steps of inflating, imaging and allowing tissue and skin to expand, until a desired aesthetic outcome is achieved. The step of allowing is preferably for a time period suitable to allow tissue/skin to expand without causing stretch marks or trauma to the tissue/skin, for example, one month to six months. Using methods of the invention, the breast can be post-operatively adjusted without additional surgeries. This is especially advantageous, for example, for a reconstruction patient (e.g. a unilateral reconstruction) in which the final aesthetic results are not usually obvious at the time of surgery due to swelling and/or capsule formation after healing.

### Example 1

### Breast Augmentation

A 29-year old woman decides to undergo elective breast augmentation surgery and desires a drastic increase in breast size. The physician suggests that she have adjustable implants placed in her breasts to allow gradual tissue expansion, which he believes will give her the chance of a rapid recovery and the best long term results.

The surgeon introduces breast implants which are no more than about fifty percent filled with saline solution, into the breasts of the woman, through small incisions and using conventional surgical techniques. The incision is closed and the breasts are allowed to heal. Sixty days after the implant procedure, the woman returns to the physician's office. The physician uses conventional fluoroscopy to produce a real-time image of the breast while the physician introduces a needle transcutaneously into the lower pole of the breast and through a lower wall of the implant. The physician carefully guides the distal end of the needle until he is assured from the fluoroscopy image that the distal end of the needle is clearly within the implant cavity and has not punctured the far wall of the implant. Using a syringe coupled to the needle, the physician then injects about 15 cc of saline into the implant, which enlarges the breast. The physician withdraws the needle, and repeats the procedure on the right breast. The woman returns to the physician 60 days later and has the same procedure done to further increase the size of her breasts.

### Example 2

### Breast Reconstruction

A 23-year old woman has had a mastectomy on her right breast to remove a malignant tumor. Seven months later, she is in remission and there is no sign that the cancer has returned. Since the surgery, the skin on her chest at the site of the mastectomy has been left flat and tight. She decides to undergo breast reconstruction surgery. The physician suggests that she undergo one-stage breast reconstruction rather than the conventional two-stage breast reconstruction in order to provide fast recovery, less down time, and the least amount of pain.

Using standard techniques, the surgeon places an adjustable, dual lumen breast implant, such as described herein, in the right chest wall, between the remaining breast tissue and the chest muscle. The implant is implanted in a substantially uninflated state, with perhaps a minimal amount of CO2 gas in the inner lumen of the implant in order to enhance visual contrast during the later imaging steps.

The incision is closed and the breast is allowed to heal. Six months later, the woman returns to the physician's office. The physician assesses the health of the woman and the condition of the implanted breast. Six months after the implant procedure, the woman returns to the physician's office. The physician's assistant applies an ultrasound probe to the patient's left breast and, while viewing an ultrasound image of the breast on an ultrasound monitor in real time, the physician introduces a needle transcutaneously into the lower pole of the breast and through a lower wall of the implant. The physician carefully guides the distal end of the needle until he is assured from the ultrasound image that the distal end of the needle is clearly within the implant cavity and has not punctured the far wall of the implant. Using a syringe coupled to the needle, the physician then injects about 10 cc of saline into the implant, which enlarges the breast without excessively stretching or traumatizing the periprosthetic tissue. The woman returns to the physician at four months intervals, and has the adjustment procedure repeated, until the reconstructed right breast is the same size as the natural left breast.

While this invention has been described with respect to various specific examples and embodiments, it is to be understood that the invention is not limited thereto and that it can be variously practiced within the scope of the invention.

What is claimed is:

1. A method of augmenting or reconstructing a breast, the method comprising the steps of:
providing a fillable breast prosthesis in a breast, the prosthesis comprising a flexible shell defining an internal cavity;
imaging the breast having the prosthesis therein;
introducing a cannula or needle through breast tissue and into the cavity of the prosthesis during the step of imaging;
guiding the needle or cannula such that a distal tip of the needle or cannula is positioned in the cavity; and
introducing into, or withdrawing a fluid from, the cavity through the distal tip of the needle or cannula during the step of imaging in order to change the volume of the implant in the breast.

2. The method of claim 1 wherein the prosthesis is a dual lumen prosthesis.

3. The method of claim 1 wherein the shell is a self sealing structure.

4. The method of claim 1 wherein the imaging comprises fluoroscopy imaging.

5. The method of claim 1 wherein the imaging comprises magnetic resonance imaging.

6. The method of claim 1 wherein the imaging comprises ultrasonic imaging.

7. The method of claim 1 for reconstructing a breast, and the step of imaging is performed using ultrasound imaging.

8. A method for implanting an adjustable gel filled mammary prosthesis and for adjusting the size of the prosthesis after implantation comprising the steps of:
providing a mammary prosthesis comprising a shell having a self sealing wall, the shell defining a cavity for containing a fluid;
implanting the prosthesis into a breast of a patient;
percutaneously introducing a distal end of a cannula through the self sealing wall;
imaging the breast, prosthesis and cannula during the step of percutaneously introducing, to assist in guiding a position of the distal end of the cannula or needle in the cavity;
introducing a fluid into the cavity through the distal end of the cannula during the step of imaging; and
withdrawing the needle or cannula from the prosthesis and breast.

9. The method of claim 8 further comprising repeating the steps of percutaneously introducing, imaging, introducing and withdrawing, until a desired breast size outcome is reached.

10. The method of claim 8 wherein the prosthesis is a dual lumen prosthesis.

11. The method of claim 8 wherein the shell is a self sealing structure.

12. The method of claim 8 wherein the imaging comprises fluoroscopy imaging.

13. The method of claim 8 wherein the imaging comprises magnetic resonance imaging.

14. The method of claim 8 wherein the imaging comprises ultrasonic imaging.

15. A breast reconstruction method for a patient who has undergone a mastectomy, the method comprising the steps of:
implanting a mammary prosthesis comprising a shell having a self sealing wall, the shell defining an inflatable cavity;
inflating the prosthesis by introducing a fluid into the cavity using a cannula inserted percutaneously into the self sealing wall of the implanted prosthesis;
imaging the prosthesis during the step of inflating;
allowing tissue/skin to expand in response to the inflated prosthesis; and
repeating the steps of inflating, imaging and allowing tissue and skin to expand, until a desired aesthetic outcome is achieved.

16. The method of claim 15 wherein the step of allowing is for a time period suitable to allow tissue/skin to expand without causing stretch marks or trauma to the tissue/skin.

17. The method of claim 15 wherein the step of imaging is done using ultrasound radiation.

18. The method of claim 1 wherein the fluid is a hydrogel.
